(19) United States
(12) Patent Application Publication
(10) Pub. No.: US 2002/0007050 A1
(43) Pub. Date: Jan. 17, 2002

(54) PROCESS FOR PREPARING ALKYL AND/OR ALKENYL OLIGOGLYCOSIDES

(76) Inventors: RAINER ESKUCHEN, LANGENFELD (DE); EIKO HASSE, DUESSELDORF (DE); BERNHARD GUTSCHE, HILDEN (DE)

Correspondence Address: COGNIS CORPORATION, 2500 RENAISSANCE BLVD., SUITE 200, GULPH MILLS, PA, 19406

(21) Appl. No.: 09/402,232

(22) PCT Filed: Mar. 30, 1998
(86) PCT No.: PCT/EP98/01851

(30) Foreign Application Priority Data
Apr. 7, 1997 (DE) 197 14 255.9

(51) Int. Cl. C07H 15/00; C08B 37/00

(52) U.S. Cl. 536/18.5; 536/18.6; 536/124

(57) ABSTRACT

A process for making alkyl and/or alkenyl oligoglycosides involving: (a) providing a glucose syrup; (b) providing a fatty alcohol; (c) combining the glucose syrup with the fatty alcohol in order to form a glucose syrup/fatty alcohol suspension; (d) providing an acidic cataylst; (e) adding the acidic catalyst to the glucose syrup/fatty alcohol suspension; and (f) acetalizing the glucose syrup/fatty alcohol suspension containing the acidic catalyst to form the alkyl and/or alkenyl oligoglycosides.

### FIELD OF THE INVENTION

This invention relates to a process for the acidic acetalization of glycoses with excess fatty alcohols using glucose sirup.

### PRIOR ART

Alkyl glucosides are important nonionic surfactants which have been adopted for use in manual dishwashing detergents and cosmetic preparations by virtue of their behavior, which in many cases corresponds to that of anionic surfactants, and their excellent ecological and dermatological properties. They can be produced by subjecting glucose or glucose sirup to acidic acetalization with an excess of higher alcohols, optionally via the intermediate stage of butyl glucosides. Corresponding industrial processes go back to the years 1968/70 [cf. U.S. Pat. No. 3,547,828, U.S. Pat. No. 3,839,318 (Rohm & Haas)]. The glucosides are normally produced discontinuously from solid glycoses, such as glucose monohydrate for example, in a stirred tank reactor with subsequent evaporation of the fatty alcohol. However, there has hitherto been no shortage of processes designed for continuous acetalization, for example using thin-layer evaporators [EP-A1 0501032 (Hüls), DE-A1 4231833 (Henkel)] or countercurrent reaction columns [EP-A1 0482325, EP-A1 0514627 (Huls)]. These processes presuppose the use of glucose sirup together with butanol because otherwise incrustation, caking and large amounts of unreacted glucose can readily occur. Unfortunately, the route involving the formation of butyl glucosides as intermediate products, which have to be subsequently reacted with fatty alcohols to form the end products, is complicated and therefore undesirable.

The problem addressed by the present invention was to provide a new process for the direct acidic acetalization of glucose sirup with fatty alcohols which would involve the use of butanol. Preferably, the process would lend itself to continuous operation.

### DESCRIPTION OF THE INVENTION

The present invention relates to a process for the production of alkyl and/or alkenyl oligoglycosides by direct acidic acetalization of glycoses with excess fatty alcohols, characterized in that suspensions of glucose sirup in fatty alcohols are used.

It has surprisingly been found that even water-containing glucose sirup can readily be acetalized with fatty alcohols both discontinuously and continuously without any need to resort to the intermediate stage of butyl glucosides, providing the glucose sirup is used in the form of a suspension in fatty alcohols.

Glucose Sirup

In the context of the present invention, the term “glucose sirup” is intended to apply to preferably refined aqueous solutions of D-glucose, maltose and higher polymers of glucose, for example oligosaccharides or dextrins, which can generally be produced by acidic hydrolysis and/or by enzymatic degradation of starch. Glucose sirups with a solids content of 50 to 85% by weight and, more particularly, 75 to 80% by weight and a DP1 degree (monomeric glucose content) of 80 to 99.9% by weight and, more particularly, 94 to 99.5% by weight, based on the solids, are particularly preferred.

Suspensions of Glucose Sirup in Fatty Alcohols

To use glucose sirup, it has to be converted before acetalization into a form which makes it easier to handle and whch, in particular, prevents decomposition occurring in the course of the reaction. To this end, it is advisable to preheat fatty alcohol to a temperature of 25 to 40° C. and to disperse the molten glucose sirup in the preheated fatty alcohol with vigorous stirring or with the aid of an inline mixer. In a particularly preferred embodiment, the glucose sirup is added to the fatty alcohol as a supercooled melt. In one alternative process, solid glucose sirup can even be dispersed. The temperature in the slurry tank should not exceed 45° C. Under the conditions mentioned, the glucose sirup precipitates as a solid and a stable glucose sirup/fatty alcohol suspension which can readily be used for the direct acetalization is formed. Accordingly, the present invention also relates to the use of suspensions of glucose sirup and fatty alcohols as raw materials for the production of alkyl and/or alkenyl oligoglycosides.

Fatty Alcohols

Fatty alcohols in the context of the present invention are primary aliphatic alcohols corresponding to formula (I)

R¹OH (I)

in which R¹ is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxosynthesis and as monomer fraction in the dimerization of unsaturated fatty alcohols. Technical fatty alcohols containing 8 to 18 carbon atoms, for example cocofatty alcohol, palm oil fatty alcohol, palm kernel oil fatty alcohol or tallow fatty alcohol, are preferred. The glycoses and the fatty alcohols are used in a molar ratio of preferably 1:1 to 1:5 and, more preferably, 1:2 to 1:3. In these ratios, the reaction components give glycosides which have a DP of 1.0 to 1.8 and preferably 1.3 to 1.7.

Catalysts

Basically, the choice of the acidic catalysts is not critical. So far as product color, foaming during acetalization, yield and the required average degree of polymerization (DP) are concerned, it has proved to be of particular advantage to use alkyl benzenesulfonic acids such as, for example, p-toluene sulfonic acid or dodecyl benzenesulfonic acid. The catalysts may be used as solutions in water or the fatty alcohols, in which case the catalyst content can be from 10 to 80% by weight. Typical catalyst contents in water are 50 to 80% by weight and, in fatty alcohols, 60 to 90% by weight. The catalysts are preferably used in quantities of 0.1 to 5% by weight and more preferably in quantities of 1 to 3% by weight, based on the starting materials.

Acetalization

The acetalization reaction can be carried out discontinuously in a stirred tank reactor. In one preferred embodiment of the invention, however, the acetalization is carried out in a cascade of 3 to 6 and preferably 3 to 4 stirred reactors. The expression “direct acetalization” means that glucose sirup and fatty alcohol are directly reacted to give the target products. In other words, the acetalization is not preceded, for example, by the preparation of a lower alkyl glycoside which then has to be trans-acetalized with the fatty alcohol. It is advisable to dry the glucose sirup/fatty alcohol suspension before the acetalization reaction. This can be done, for example, by designing the first reactor of the cascade as a drying stage because the presence of water in the mixture can lead to an unwanted increase in the polysugar content of the end product and to caking on the walls of the reactor. Basically, drying can be carried out both in batches and continuously. In batch drying, the temperature should be increased in steps whereas, in continuous drying, the addition of the glucose sirup/fatty alcohol slurry should preferably be adjusted in such a way that the water content in the drying reactor is below 0.5% by weight. Since a uniformly high temperature in the reaction stage can also lead to an increase in the polysugar content, it is also advisable to use a reactor cascade and to apply a temperature gradient of 70 to 1 20° C. and/or to carry out the reaction under reduced pressure. A pressure gradient of, for example, 20 to 50 mbar may also be applied or, alternatively, different catalyst concentrations may be adjusted in the individual reactors. On completion of the acetalization reaction, it is advisable to neutralize the reaction products by addition of aqueous bases, for example sodium hydroxide and/or magnesium oxide, before the excess fatty alcohol is removed in known manner by distillation.

If necessary, however, fatty alcohol may also be added in addition to the fatty alcohol added as a suspension containing the glucose sirup.

### EXAMPLES

Preparation of the Glucose Sirup/Fatty Alcohol Suspension.

In a stirred tank reactor, cocofatty alcohol was heated to 40° C. and mixed with vigorous stirring with glucose sirup, the temperature being kept below 45° C. and the glucose sirup being added as a supercooled melt at 30° C. The glucose sirup was precipitated as a solid and was processed in situ to a glucose sirup/fatty alcohol suspension.

### Example 1

454 g of the suspension were introduced into a I liter stirred reactor, heated for 1 hour at 35 mbar to 75° C. and dried. The catalyst solution was then added, the mixture was heated to the reaction temperature and was kept at that temperature until the residual glucose content had fallen to 0.75% by weight.

### Example 2

454 g of the suspension were introduced into the drying reactor R1 of a reactor cascade consisting of four 1-liter glass reactors and dried as described in Example 1. The other reactors were filled with fatty alcohol. The reactors were thermostatted by circulation thermostats containing heat transfer oil. A rotary slide-valve oil pump was used as the vacuum pump. The catalyst solution was introduced into reactor R2 by another pump. The reaction mixture flowed from reactor R4 into a receiving flask in which it was neutralized with aqueous sodium hydroxide solution (25% by weight).

The test data and results of the two Examples are set out in Table 1.

TABLE 1
Production of alkyl glucosides

| | 1 (quantities in g) | 2 (quantities in g/h) |
|---|---|---|
| Glucose sirup (70% by weight) | 114 | 114 |
| Cocofatty alcohol | 340 | 340 |
| Dodecyl benzenesulfonic acid* | 1.1 | 1.1 |
| Number of reactors (total) | 1 | 4 |
| Pressure [mbar] | 35 | 35 |
| Temperature R1 [° C.] | 105 | 75 |
| Temperature R2 [° C.] | — | 105 |
| Temperature R3 [° C.] | — | 105 |
| Temperature R4 [° C.] | — | 105 |
| Residual glucose in the product | 0.75 | 0.5 |
| [% by weight] | | |
| Polymer content in the product | 3.6 | 3.2 |
| [% by weight] | | |
| DP** | 1.42 | 1.42 |

*) 70% by weight in cocofatty alcohol, **) degree of polymerization

1. A process for the production of alkyl and/or alkenyl oligoglycosides by direct acidic acetalization of glycoses with excess fatty alcohols, characterized in that suspensions of glucose sirup in fatty alcohol are used.

2. A process as claimed in claim 1, characterized in that fatty alcohols corresponding to formula (I):
R¹OH (I)
in which R¹ is an aliphatic, linear or branched hydrocarbon radical containing 6 to 22 carbon atoms and 0 and/or 1, 2 or 3 double bonds are used.

3. A process as claimed in claims 1 and 2, characterized in that the glucose sirup and the fatty alcohols are used in a molar ratio of 1:1 to 1:10.

4. A process as claimed in claims 1 to 3, characterized in that alkyl benzenesulfonic acids are used as catalysts.

5. A process as claimed in claims 1 to 4, characterized in that the catalysts are used in quantities of 0.1 to 5% by weight, based on the starting materials.

6. A process as claimed in claims 1 to 5, characterized in that the acetalization is carried out continuously in a cascade of 3 to 6 reactors.

7. A process as claimed in claim 6, characterized in that a temperature gradient of 70 to 1 20° C. is applied to the reactor cascade for the acetalization reaction.

8. A process as claimed in claims 1 to 7, characterized in that the reaction is carried out under reduced pressure.

9. A process as claimed in claims 1 to 8, characterized in that the glucose sirup is dried before the acetalization reaction.

10. The use of suspensions of glucose sirup and fatty alcohols as raw materials for the production of alkyl and/or alkenyl oligoglycosides.
